SEQUENCE LISTING (8 sequences)

SEQ ID NO: 1 - DNA, 36 bp, Artificial sequence
  Primer
gcgaagcttgccaccatggtgggcatctggaccctc

SEQ ID NO: 2 - DNA, 36 bp, Artificial sequence
  Primer
gacacaacatttgcgctcgttagatctggatccttc

SEQ ID NO: 3 - DNA, 24 bp, Artificial sequence
  Primer
gagcgcaaatgttgtgtcgagtgc

SEQ ID NO: 4 - DNA, 36 bp, Artificial sequence
  Primer
attaagcattctagatcatttacccggagacaggga

SEQ ID NO: 5 - DNA, 33 bp, Artificial sequence
  Primer
atcggatccagcatggctgacaagatcctgagg

SEQ ID NO: 6 - DNA, 32 bp, Artificial sequence
  Primer
cggcctcgagcatcatctaaggaagtattggc

SEQ ID NO: 7 - DNA, 31 bp, Artificial sequence
  Primer
gcgaagcttacacgaccaatatcgattacta

SEQ ID NO: 8 - DNA, 30 bp, Artificial Sequence
  Primer
cgccatggttaacaattagttgtcggagag

(19) United States
(12) Patent Application Publication
(10) Pub. No.: US 2007/0207160 A1
(43) Pub. Date: Sep. 6, 2007

(54) Apoptosis inhibitors

(76) Inventors: Peter Krammer, Heidelberg (DE); Michael Westendorp, Heidelberg (DE); Klaus Schulze-Osthoff, Dossenheim (DE); Klaus-Michael Debatin, Heidelberg (DE); Rainer Frank, Heidelberg (DE); Jens Dhein, Edingen-Nhsn. (DE); Henning Walczak, Heidelberg (DE); Eckart Knipping, Heidelberg (DE); Kirstin Stricker, Heidelberg (DE)

Correspondence Address: NIXON PEABODY, LLP, 401 9TH STREET, NW, SUITE 900, WASHINGTON, DC, 20004-2128, US

(21) Appl. No.: 11/705,956

(22) Filed: Feb. 14, 2007

Related U.S. Application Data
(62) Division of application No. 08/564,331, filed on Apr. 6, 1999, now abandoned.

(30) Foreign Application Priority Data
Apr. 8, 1994 (DE) P 44 12 177.6

(51) Int. Cl. A61K 39/00

(52) U.S. Cl. 424/178.1

(57) ABSTRACT

This invention relates to compositions for inhibiting apoptosis, containing one to all components of: (a) an APO-1-inhibiting compound; (b) a compound inhibiting and catching, respectively, the APO-1 ligand; and (c) a compound inhibiting the intracellular APO-1 signalling pathway, the component(s) being not considered foreign in an individual, as well as conventional excipients. Furthermore, this invention relates to compounds suitable for inhibiting apoptosis and having at least one extracellular APO-1 domain and a carrier, the domain(s) and the carrier being not considered foreign in an individual.

### I. FIELD OF THE INVENTION

The present invention relates to compositions for inhibiting apoptosis. Furthermore, the present invention concerns compounds suitable for inhibiting apoptosis as well as processes for the production thereof.

### II. BACKGROUND OF THE INVENTION

Apoptosis is the designation for programmed cell death. It is found in many physiological processes, including organ development, metamorphosis, cataplasia and tumor regression. Apoptosis is accompanied by a condensation of cytoplasm, a loss of plasma membrane villi, a segmentation of the nucleus and extensive decomposition of chromosomal DNA. Oehm et al, 1992, The Journal of Biological Chemistry 267:10709-10715.

Cells which are positive for apoptosis often include a cellular surface protein referred to as APO-1. APO-1 is a glycoprotein related to the tumor necrosis factor/nerve growth factor-receptor family. By cross-linking APO-1 via an anti-APO-1 antibody, apoptosis is induced in said cells. Oehm et al, supra. It seems that the same can also be effected by binding a soluble or membrane-bound protein referred to as APO-1 ligand to APO-1. Suda and Nagata, 1994, Exp. Med., The Rockefeller University Press 179:873-879.

Prior to the subject invention, nothing was known about the inhibition of apoptosis. Applicant, however, recently made the unexpected discovery that apoptosis occurs in a number of severe diseases, including AIDS and autoimmune diseases. For example in AIDS, it seems that the vast decrease of the CD4-T cells is driven by apoptosis. Consequently, an understanding of factors which suppress the programmed cell death would provide significant advancement for the treatment of major diseases.

Thus, it is the object of the present invention to provide a composition by means of which the programmed cell death can be inhibited.

### III. SUMMARY OF THE INVENTION

The present invention is directed to compounds and compositions for inhibition of apoptosis. The target for inhibition of apoptosis is the transmembrane glycoprotein APO-1 and its signalling pathway, which has been shown to be involved in apoptosis. The present invention offers a new way of treating diseases in which apoptosis of special cells plays an important role, including AIDS.

In one embodiment, the invention is directed to compounds suitable for inhibiting apoptosis. For this purpose, it can be employed as such or in combination with one to all components of:
(a) An APO-1-inhibiting compound;
(b) Another compound inhibiting and catching, respectively, the APO-1 ligand; and
(c) A compound inhibiting the intracellular APO-1 signalling pathway, the component(s) being not considered foreign in an individual.

In a second embodiment, the invention is directed to a compound in particularly for inhibiting apoptosis in a disease associated with an HIV infection. For this purpose, the compound according to the invention can be used as such or in combination with one to all components of:
(a) An APO-1-inhibiting compound;
(b) Another compound inhibiting and catching, respectively, the APO-1 ligand;
(c) A compound inhibiting the intracellular APO-1 signalling pathway;
(d) A compound inhibiting the TAT receptor;
(e) A compound inhibiting and catching; respectively, TAT;
(f) A compound inhibiting the intracellular TAT receptor signalling pathway;
(g) A compound inhibiting the CD4 receptor;
(h) A compound inhibiting and catching, respectively, gp 120; and
(i) A compound inhibiting the intracellular CD4 receptor signalling pathway, the component(s) being not considered foreign in an individual.

The present invention is further directed to processes for the production of apoptosis inhibiting compounds and compositions and the testing thereof.

### IV. BRIEF DESCRIPTION OF THE DRAWINGS

FIG. 1 depicts the inhibition of apoptosis induced in SKW6.4 cells by means of anti-APO-1 antibodies (20 ng/ml) by human APO-1-Ig and anti-APO-1F(ab′)₂, respectively.

FIG. 2A depicts the stimulation of the proteolytic activity of ICE by an anti-APO-1 antibody.

FIG. 2B depicts the inhibition of apoptosis by DCI.

FIG. 2C depicts the inhibition of apoptosis by YVAD-CHO.

FIG. 2D depicts the inhibition of apoptosis by anti-sense ICE- and CrmA-cDNA, respectively.

### V. DETAILED DESCRIPTION OF THE INVENTION

A. Compositions for Inhibition of Apoptosis

The invention provides compositions for the inhibition of apoptosis which contain one or several of the following components:
(a) An APO-1-inhibiting compound;
(b) A compound inhibiting and catching, respectively, the APO-1 ligand; and
(c) A compound inhibiting the intracellular APO-1 signalling pathway;
provided that the component(s) being not considered foreign in an individual, as well as conventional excipients.

The expression “APO-1-inhibiting compound” comprises any compound suitable for inhibiting APO-1. Preferably, this is a blocking non-cytotoxic anti-APO-1 antibody or an anti-APO-1 antibody without Fc portion, e.g., an F(ab)-, F(ab)₂- or Fᵥ fragment of an anti-APO-1 antibody. Such a fragment is prepared as usual, a person skilled in the art using e.g., the anti-APO-1 antibody described in Oehm et al, supra, or anti-APO-1-F(ab)₂ fragment described in Dhein et al., 1992, The Journal of Immunology 149:3166-3173, as a basis. The latter can also be used directly.

The lack of the Fc portion in the above antibody prevents the cross-linking of bound APO-1 and thus the induction of apoptosis. Surprisingly, APO-1 is inhibited by such an antibody.

An APO-1-ligand analogue has to be mentioned as another preferred “APO-1-inhibiting compound”. It still binds to APO-1, but no longer induces the intracellular APO-1 signalling pathway. Such an analogue is prepared as usual, the person skilled in the art using e.g., the APO-1 ligand described in Suda and Nagata, supra, as a basis.

The expression “compound inhibiting and catching, respectively, APO-1 ligand” comprises any compound suitable for inhibiting and catching, respectively, the APO-1 ligand. This is preferably one of the following compounds:
An anti-APO-1 ligand antibody;
An APO-1;
An extracellular APO-1 domain;
A compound having at least one extracellular APO-1 domain and a carrier;
A peptide having an APO-1 ligand binding site; and
A compound at least one peptide having an APO-1 ligand binding site and a carrier.

Such a compound is prepared as usual. For the production of an anti-APO-1 ligand antibody, the person skilled in the art will use, e.g., the APO-1 ligand described in Suda and Nagata, supra, as a basis. The skilled artisan will pay attention to the fact that the Fc portion of the antibody is not considered foreign in an individual. The skilled artisan is familiar with processes enabling this. Furthermore, the person skilled in the art will use, e.g., the APO-1 and its extracellular domain, respectively, described in EP-92 107 060.3 as a basis for the production of APO-1 and an extracellular APO-1 domain, respectively. In addition, the skilled artisan will utilize, e.g., the combination of the above-mentioned APO-1 ligand and the above-mentioned APO-1 and its extracellular domain, respectively, for the production of a peptide having an APO-1 ligand binding site.

The production of a compound having at least one extracellular APO-1 domain and a carrier is described by way of example below. It is possible to prepare analogously a compound having at least one peptide an APO-1 ligand binding site and a carrier.

The expression “a compound inhibiting the intracellular APO-1 signalling pathway” comprises any compound suitable for inhibiting the intracellular APO-1 signalling pathway. This is preferably one of the following compounds:
An inhibitor of the “interleukin-1β converting enzyme” (ICE), particularly 3,4-dichloroisocoumarin (DCI), YVAD-CHO, and ICE-specific tetrapeptide, or CrmA, a protein of vaccinia virus and derivatives thereof, respectively. It may be favorable to use YVAD-CHO or CrmA and derivatives thereof, respectively, as expressible nucleic acids. Furthermore, it may be favorable to use an ICE-anti-sense nucleic acid and a derivative thereof, respectively, as the ICE inhibitor.
An inhibitor of proteases structurally related to ICE, particularly of Nedd-2/Ich-1 or priCE.

Such an inhibitor compound is prepared as usual. For the production of DCI and a derivative thereof, respectively, the person skilled in the art will return, e.g., to the study by Harper et al, 1985, Biochemistry 24:1831-1841. Furthermore, the skilled artisan will consider the study by Thornberry et al, 1992, Nature 356:768-774, as regards YVAD-CHO and a derivative thereof, respectively. In addition, the skilled artisan will consider e.g. the study by Ray et al., 1994, Cell 69:597-604, regarding CrmA or a derivative thereof. Besides, the skilled artisan will consult, e.g., the study by Cerretti et al., 1992, Science 256:97-100, regarding the ICE-anti-sense nucleic acid and a derivative thereof, respectively. Moreover, the skilled artisan will return to the study by Wang et al, 1994, Cell 78:739-750 and by Lazebnik et al, 1994, Nature 371:346-347, respectively, as regards the proteases Nedd-2/Ich-1 and priCE, respectively.

B. Compositions for Inhibition of Apoptosis for the Treatment of AIDS

According to the invention the above-mentioned compositions are used particularly for inhibiting apoptosis in a disease associated with an HIV infection. In this connection, this composition will prove to be especially advantageous if it furthermore contains one to all components of:
(a) A compound inhibiting the TAT-receptor,
(b) A compound inhibiting and catching, respectively, TAT,
(c) A compound inhibiting the intracellular TAT receptor signalling pathway,
(d) A compound inhibiting the CD4 receptor,
(e) A compound inhibiting and catching, respectively, gp 120; and
(f) A compound inhibiting the intracellular CD4 receptor signalling pathway, the component(s) being not considered foreign in an individual.

The expression “a compound inhibiting the TAT receptor” comprises any compound suitable for inhibiting the TAT receptor. This is preferably an anti-TAT-receptor antibody. It is prepared as usual. The person skilled in the art will pay attention to the fact that the Fc portion of the antibody is not considered foreign in an individual. The person skilled in the art is familiar with processes enabling this.

A TAT analogue has to be mentioned as another preferred “compound inhibiting the TAT receptor”. It still binds to the TAT receptor but no longer induces the intracellular TAT receptor signalling pathway. Such an analogue is prepared as usual, the person skilled in the art using e.g. the TAT described in Arya et al, 1985, Science 229:69-73, as a basis.

The expression “a compound inhibiting and catching, respectively, TAT” comprises any compound suitable for inhibiting and catching, respectively, TAT. This is preferably one of the following compounds:
An anti-TAT antibody;
A TAT receptor;
An extracellular TAT receptor domain;
A compound having at least one extracellular TAT receptor domain and a carrier;
A peptide having a TAT binding site;
A compound having at least one peptide having a TAT binding site and a carrier;
A TAT binding site on a nucleic acid basis; and
A transdominant TAT mutant.

Such a compound is prepared as usual. For the production of an anti-TAT antibody, the person skilled in the art will use e.g. the TAT described in Arya et al., supra, as a basis. The skilled artisan will pay attention to the fact that the Fc portion of the antibody is not considered foreign in an individual, as he is familiar with processes enabling this. Furthermore, the skilled artisan will use, e.g., the TAT-LTR sequence described in Feng and Holland, 1988, Nature 334:165-167, as a basis regarding a TAT binding site on a nucleic acid basis. Besides, the person skilled in the art will use, e.g., the mutant described in Echetebu and Rice, 1993, J. Acquir. Immune Def. Syndrome 6:550-557, as a basis regarding a transdominant TAT mutant.

The expression “a compound inhibiting the intracellular TAT-receptor signalling pathway” comprises any compound suitable for inhibiting the intracellular TAT receptor signalling pathway.

The expression “a compound inhibiting the CD4 receptor” comprises any compound suitable for inhibiting the CD4 receptor. This is preferably an anti-CD4 receptor antibody. It is prepared as usual, the person skilled in the art using, e.g., the CD4 receptor described in Capon and Ward, 1991, Annu. Rev. Immunol. 9:649-678, as a basis. The person skilled in the art will pay attention to the fact that the Fc portion of the antibody is not considered foreign in an individual. The person skilled in the art is familiar with processes enabling this.

A gp 120 analogue has to be mentioned as another preferred “compound inhibiting the CD4 receptor”. It still binds to the CD4 receptor but no longer induces the intracellular CD4 receptor signalling pathway. Such an analogue is prepared as usual, the person skilled in the art using e.g. the gp 120 described in Capon and Ward, supra, as a basis.

The expression “a compound inhibiting and catching, respectively, gp 120” comprises any compound suitable for inhibiting and catching, respectively, gp 120. This is preferably one of the following compounds:
an anti-gp 120 antibody;
A CD4 receptor;
An extracellular CD4 receptor domain;
A compound having at least one extracellular CD4 receptor domain and a carrier;
A peptide having a gp 120 binding site; and
A compound having at least one peptide having a gp 120 binding site and a carrier.

Such a compound is prepared as usual. For the production of an anti-gp 120 antibody, the person skilled in the art will use e.g. the gp 120 described in Capon and Ward, supra, as a basis. The skilled artisan will pay attention to the fact that the Fc portion of the antibody is not considered foreign in an individual, as he is familiar with processes enabling this. Furthermore, the person skilled in the art will use the CD4 receptor and its extracellular domain, respectively, described in Capon and Ward, supra, as a basis regarding the production of a CD4 receptor and an extracellular CD4 receptor domain, respectively. In addition, the skilled artisan will utilize, e.g., the combination of the above-mentioned gp 120 and the above-mentioned CD4 receptor and its extracellular domain, respectively, for the production of a peptide having a gp 120 binding site.

The production of a compound having at least one extracellular CD4 receptor domain and a peptide having at least one gp 120 binding site, respectively, and a carrier can be carried out analogously as described below for a compound having at least one extracellular APO-1 domain and a carrier.

The expression “a compound inhibiting the intracellular CD4 receptor signalling pathway” comprises any compound suitable for inhibiting the intracellular CD4 receptor signalling pathway.

It is understood that an above-mentioned composition may have one to several compounds of an individual component.

According to the invention a compound having at least one extracellular APO-1 domain and a carrier is also provided, the domain(s) and the carrier being not considered foreign in an individual.

The expression “carrier” comprises any compound to which one or more extracellular APO-1 domains may be bound.

In a preferred embodiment, the carrier is a protein, e.g., serum albumin, hemoglobin, fibrinogen, collagen or an Fc portion of an antibody, the latter being preferred. In an especially preferred embodiment, the compound according to the invention is a fusion protein.

C. Production of the Compositions

A compound according to the invention can be prepared as usual. In the case of a fusion protein, the following production process proves to be favorable.
(a) Amplification of a DNA by conventional PCR technique, the DNA encoding for at least one extracellular APO-1 domain and a binding region attached to the 3′ end thereof;
(b) Amplification of a DNA by conventional PCR technique, the DNA encoding for a protein carrier and a binding region attached to the 5′ end of the protein carrier;
(c) Combination of the amplified DNAs of (a) and (b) and further common amplification thereof by conventional PCR technique, a primer corresponding to the 5′ end of the DNA of the APO-1 domain and a primer corresponding to the 3′ end of the DNA of the protein carrier being used so as to obtain an amplified DNA fragment containing both DNAs in fusion; and
(d) Insertion of the DNA fragment of (c) in an expression vector and expression of the DNA fragment as usual.

In a preferred embodiment, the binding region of (a) and (b) is an antibody hinge region or a portion thereof. Furthermore, it may also be a thrombin cleaving site.

In the above-mentioned production process, various DNAs are amplified by conventional PCR technique. The person skilled in the art will use as a basis, e.g., the DNA described in EP-92 107 060.3, supra, as the DNA encoding for at least one extracellular APO-1 domain. The skilled artisan will attach a DNA encoding for a binding region to the 3′ end thereof. In the case of a DNA encoding for a hinge region of a human antibody or a portion thereof, the person skilled in the art will return, e.g., to the DNA described in Dübel et al, 1992, Methods in Molecular and Cellular Biology 3:47-52. As regards the DNA encoding for the protein carrier, e.g., Fc portion of a human antibody, the person skilled in the art will also return, e.g., to the DNA described in Dübel, supra.

The amplified DNA fragment is expressed in conventional vectors such as pCDN83, pCEV4 and pCDM8 for the expression in animal cells, pGEMEX and pUC for the expression in E. coli, as well as pY100 and YCpAD1 for the expression in yeast. L, COS and CHO cells are especially suitable as animal cells while particularly E. coli strains have to be mentioned as procaryotic microorganisms and particularly those of saccharomyces and Pichia pastoris have to be indicated as yeast cells.

D. Use of the Compositions

A compound according to the invention is extremely well suitable for inhibiting apoptosis. For this purpose, it can be employed as such or in combination with one to all components of:
(a) An APO-1-inhibiting compound;
(b) Another compound inhibiting and catching, respectively, the APO-1 ligand; and
(c) A compound inhibiting the intracellular APO-1 signalling pathway, the component(s) being not considered foreign in an individual.

Reference is made to the above statements on the individual components (a), (b) and (c). These statements apply here correspondingly.

A compound according to the invention is particularly suitable for inhibiting apoptosis in a disease associated with an HIV infection. For this purpose, the compound according to the invention can be used as such or in combination with one to all components of:
(a) An APO-1-inhibiting compound;
(b) Another compound inhibiting and catching, respectively, the APO-1 ligand;
(c) A compound inhibiting the intracellular APO-1 signalling pathway;
(d) A compound inhibiting the TAT receptor;
(e) A compound inhibiting and catching; respectively, TAT;
(f) A compound inhibiting the intracellular TAT receptor signalling pathway;
(g) A compound inhibiting the CD4 receptor;
(h) A compound inhibiting and catching, respectively, gp 120; and
(i) A compound inhibiting the intracellular CD4 receptor signalling pathway, the component(s) being not considered foreign in an individual.

As regards the individual components (a)-(i), reference is made to the above statements, components (d)-(i) being referred to as (a)-(f) above. The above statements apply here accordingly.

The present invention offers a new way of treating diseases in which apoptosis of special cells plays an important part. As such, the present invention represents a break-through particularly for the treatment of AIDS.

The following preparations and examples are given to enable those skilled in the art to more clearly understand and to practice the present invention. The present invention is not limited in scope by the exemplified embodiments, which are intended as illustrations of single aspects of the invention only, and methods which are functionally equivalent are within the scope of the invention. Indeed, various modifications of the invention in addition to those described herein will become apparent to those skilled in the art from the foregoing description and accompanying drawings. Such modifications are intended to fall within the scope of the appended claims.

### VI. EXAMPLES

### A. Example 1

### Production of a Fusion Protein in Which an Extracellular APO-1 Domain is Fused Via an Antibody-Hinge Region to an Fc Portion of a Human Antibody

For the preparation of the above-mentioned fusion protein, a cDNA encoding an extracellular APO-1 domain (Oehm et al, supra) and a cDNA encoding an Fc portion of a human antibody (Dübel et al, supra) were subjected to conventional PCR amplification.

The primers used for the cDNA of the extracellular APO-1 domain were:

(SEQ ID NO:1)

5′ GCG AAG CTT GCC ACC ATG GTG GGC ATC TGG ACC
CTC 3′,

whereby a Hind III site is introduced upstream of a complete Kozak-Consensus region surrounding the initiator methionine; and

(SEQ ID NO:2)

5′ GAC ACA ACA TTT GCG CTC GTT AGA TCT GGA TCC
TTC 3′,

which encodes for the 3′ end of the extracellular APO-1 domain and the first 18 bp of the hinge region.

The primers used for the cDNA of the Fc portion were:

(SEQ ID NO:3)

5′ GAG CGC AAA TGT TGT GTC GAG TGC 3′,

which encodes for the first 18 bp of the hinge region and the 5′ end of the Fc portion; and

(SEQ ID NO:4)

5′ ATT AAG CAT TCT AGA TCA TTT ACC CGG AGA CAG GGA
3′,

which encodes for the 3′ end of the Fc portion and introduces an XbaI site downstream of the stop codon.

The resulting PCR products were separated in a low-melting-point agarose gel and the gel pieces which included properly sized DNA molecules were combined. Another PCR amplification was carried out with a sample thereof. The primers used were only those which correspond to the 5′ end of the extracellular APO-1 domain and the 3′ end of the Fc portion, respectively. Hence it was possible to fuse the extracellular APO-1 domain via the common hinge region to the Fc portion. A “fused” DNA fragment was obtained.

This fragment was cleaved with HindIII and XbaI, purified over an agarose gel and cloned in the vector pCDM8. The sequence of the insert of pCDM8 was determined by dideoxynucleotide sequencing.

### B. Example 2

### Inhibition of Apoptosis by Human APO-1-Ig and Anti-APO-1F(ab′)₂

SKW 6.4 cells (Oehm et al, supra) are apoptosis-positive cells, i.e. apoptosis can be induced therein, e.g. by binding an anti-APO-1 antibody.

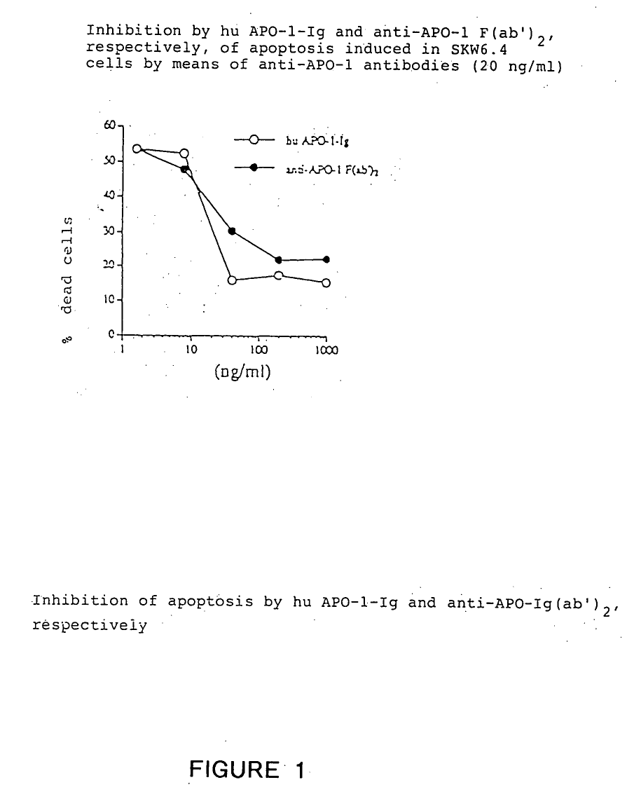

SKW 6.4 cells were incubated with an anti-APO-1 antibody in the presence of varying amounts of the fusion protein of Example 1 (human APO-1-Ig) and of anti-APO-1-F(ab)₂ (see above), respectively, for 24 hours. The inhibition of apoptosis was determined. See, FIG. 1.

It turned out that the fused portion of Example 1 and anti-APO-1-F(ab)₂ have a strong apoptosis-inhibiting effect. It is especially strong in the fusion protein.

### C. Example 3

### Inhibition of apoptosis by DCI, YVAD-CHO, Anti-Sense-ICE- and CrmA-cDNA, Respectively

L 929-APO-1 cells (Schulze-Osthoff et al., 1994, EMBO J. 13:4587-4589) are apoptosis-positive like the SKW 6.4 cells of Example 2, supra.

1. Stimulation of the Proteolytic Activity of ICE by an Anti-APO-1 Antibody

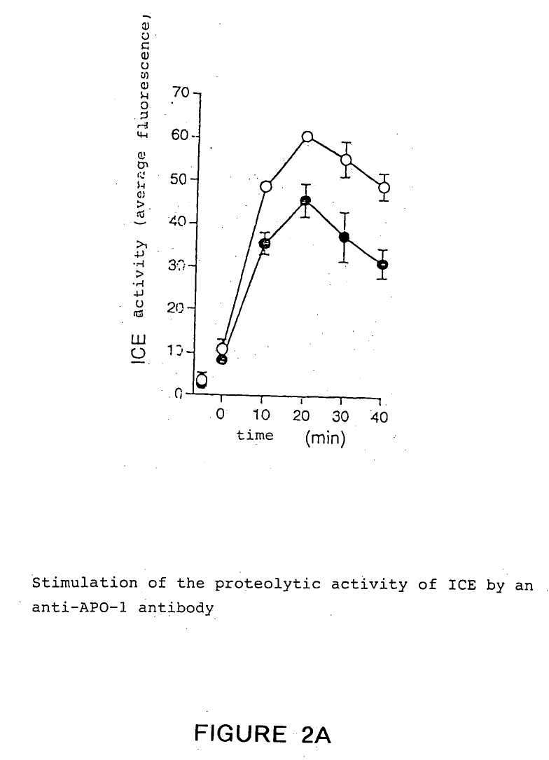

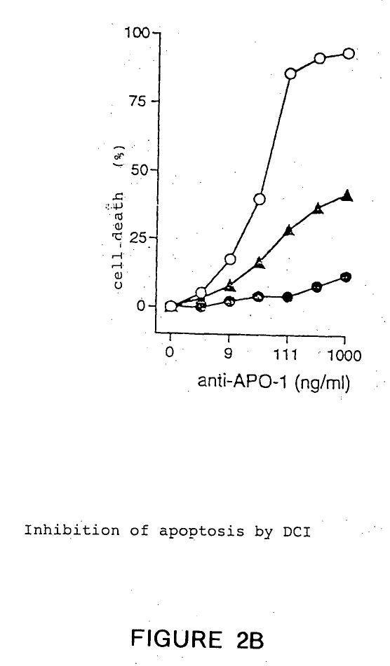

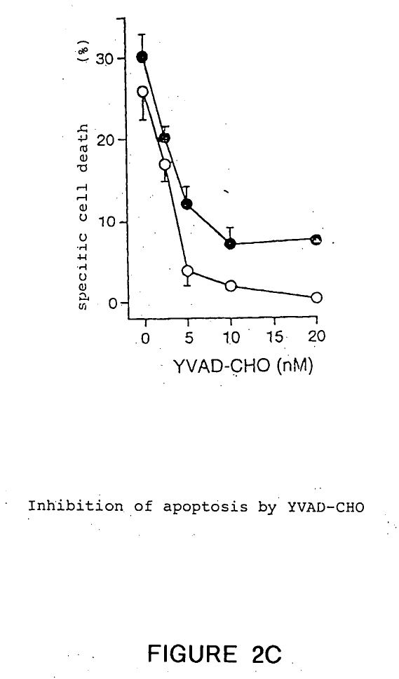

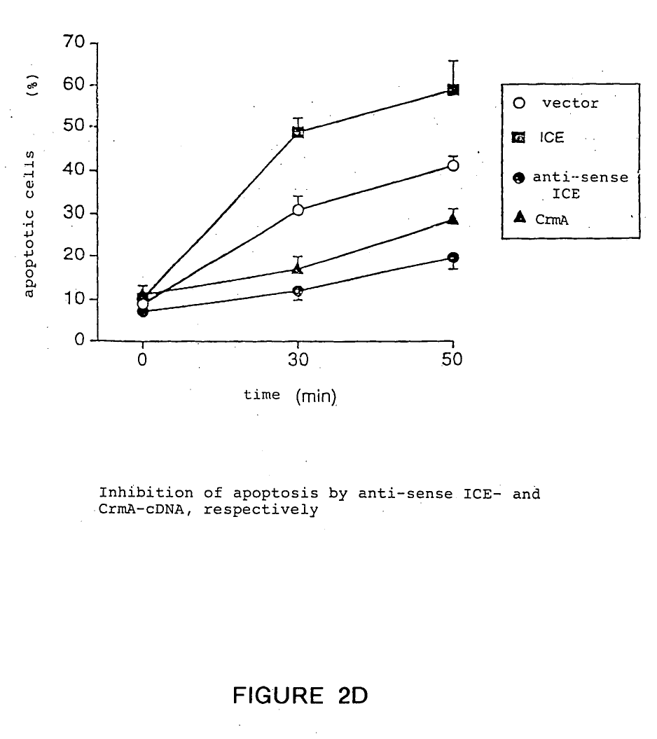

L 929-APO-1 cells (◯) and SKW 6.4 cells (●) were cultivated and treated with anti-APO-1 antibodies (1 μg/ml) for the periods indicated in FIG. 2A 10 minutes before the cells were harvested, they were permeabilized using 0.05% digitonin and incubated with 20 μM of the fluorogenic ICE substrate DABCYL-YVADAP-EDANS (Bachem, Bubendorf, Switzerland). The cells were harvested and analyzed by FACS analysis using an excitation wavelength of 360 nm and an emission wavelength of 488 nm.

It showed that the proteolytic activity of ICE can be stimulated by an anti-APO-1 antibody.

2. Inhibition of Apoptosis Induced by an Anti-APO-1 Antibody Using DCI

L 29-APO-1 cells were incubated with DCI (●45μM, ▴15 μM) or without DCI (◯). After one hour, anti-APO-1 antibody was added in the amounts indicated in FIG. 2B and allowed to remain on the cells for 7 hours. The percentage cell death was determined by formazan production from diphenyltetrazolium salt (MTT assay).

It showed that apoptosis induced by an anti-APO-1 antibody can be inhibited by DCI.

3. Inhibition of Apoptosis Induced by an Anti-APO-1 Antibody Using YVAD-CHO

L 29-APO-1 cells (◯) and SKW 6.4 cells (●) were permeabilized by a short hypotonic shock and incubated with the indicated concentrations of YVAD-CHO indicated in FIG. 2C for 30 minutes. The cells were incubated with anti-APO-1 antibodies (1 μg/ml) for 60 minutes. Apoptosis was determined by measuring a DNA fragmentation using the Hoechst dye 33342 (Molecular Probes Eugene, Oreg.). Data of specific cell death (cell death in the presence of an anti-APO-1 antibody minus cell death in the absence of such an antibody) was obtained from triple measurements by using an FACS Vantage flow cytometer. Spontaneous cell death in the absence of an anti-APO-1 antibody was less than 7%.

It showed that apoptosis induced by an anti-APO-1 antibody can be inhibited by YVAD-CHO.

4. Inhibition of Apoptosis Induced by an Anti-APO-1 Antibody Using Anti-Sense ICE- and CrmA-cDNA

The following expression plasmids were prepared:

pCAGGS-ICE. A mouse ICE cDNA was isolated by RT-PCR by using EL-4/c mRNA and oligo dT primers for the first strand synthesis. A 1322 bp PCR product was obtained by the primer pair

(SEQ ID NO:5)

5′-ATCGGATCCAGCATGGCTGACAAGATCCTGAGG-3′;
and

(SEQ ID NO:6)

5′-CGGCCTCGAGCATCATCTAAGGAAGTATTGGC-3′,

which was used as sample for screening a E 14/13 cDNA expression colony bank cloned in pCAGGS. Niwa et al, 1994, Gene 108:193-200. A 1387 bp ICE cDNA clone was obtained whose sequence was determined by DNA sequencing. This clone was referred to by pCAGGS-ICE.

PCAGGS-anti-senseICE. A 320 bp EcoRI fragment of the above-mentioned ICE cDNA was cloned in pCAGGS in reverse orientation, the fragment containing 48 bp of 5′ UTR and the first 255 bp of ICE-ORF. The expression plasmid pCAGGS-anti-sense ICE was obtained.

pSV25S-CrmA. In a conventional PCR, a cDNA encoding for CrmA was obtained from vaccinia virus DNA by using the primer pair

(SEQ ID NO:7)

5′-GCGAAGCTTACACGACCAATATCGATTACTA-3′;
and

(SEQ ID NO:8)

5′-CGCCATGGTTAACAATTAGTTGTCGGAGAG-3′.

This cDNA was cloned as HindIII/KpnI fragment into the known plasmid pSV25S. The expression plasmid pSV25S-CrmA was obtained.

The above expression plasmids were used for the transfection of L 929-APO-1 cells. For this purpose, the cells were taken up in TBS buffer and equilibrated on ice for 10 minutes. The cells were transfected with 20 μg of the above expression plasmids each by using a Biorad electroporator (960 μFD, 220 V). After the electroporation, the cells were held on ice for another 30 minutes before they were sowed in cell culture plates. Dead cells were removed by a washing step after 16 hours. Living cells were treated with anti-APO-1 antibody (1 μg/ml) for the periods indicated in FIG. 2D. As described above, apoptosis was determined by FACS analysis using the Hoechst dye 33342. Data was determined as percentage cell death from double experiments.

It showed that apoptosis induced by an anti-APO-1-antibody can be inhibited by anti-sense ICE and CrmA, respectively.

All references cited within the body of the instant specification are hereby incorporated by reference in their entirety.

1-20. (canceled)

21. A method for inhibiting apoptosis in a patient in need thereof, comprising administering to said patient a therapeutically effective amount of a fusion protein comprising the extracellular domain of human APO-1 and an Fc portion of a human antibody, and a suitable excipient.

22. The method of claim 21, wherein said Fc portion is fused to amino acid residue 173 of said extracellular domain of human APO-1.

23. The method of claim 21, wherein said Fc portion is fused to amino acid residue 171 of said extracellular domain of human APO-1.

24. The method of any of claims 21, 22, or 23, wherein said patient has a disease associated with HIV infection.

25. The method of any of claims 21, 22, or 23, wherein said patient has an autoimmune disorder.

26. A method for inhibiting apoptosis in a patient in need thereof, comprising administering to said patient a therapeutically effective amount of a fusion protein comprising (1) the extracellular domain of human APO-1 and human serum albumin, (2) the extracellular domain of human APO-1 and human fibrinogen, (3) the extracellular domain of human APO-1 and hemoglobin, or (4) the extracellular domain of human APO-1 and collagen, and a suitable excipient.

27. The method of claim 26, wherein said Fc portion is fused to amino acid residue 173 of said extracellular domain of human APO-1.

28. The method of claim 26, wherein said Fc portion is fused to amino acid residue 171 of said extracellular domain of human APO-1.

29. The method of any of claims 26, 27, or 28, wherein said patent has a disease associated with HIV infection.

30. The method of any of claims 26, 27, or 28, wherein said patient has an autoimmune disorder.

31. A method for inhibiting apoptosis in a cell, comprising treating said cell with an effective amount of a fusion protein comprising the extracellular domain of human APO-1 and an Fc portion of a human antibody.

32. The method of claim 31, wherein said Fc portion is fused to amino acid residue 173 of said extracellular domain of human APO-1.

33. The method of claim 31, wherein said Fc portion is fused to amino acid residue 171 of said extracellular domain of human APO-1.

34. The method of any of claims 31, 32, or 33, wherein said cell is affected by an HIV infection.

35. The method of any of claims 31, 32, or 33, wherein said cell is affected by an autoimmune disorder.

36. A method for inhibiting apoptosis in a cell, comprising treating said cell with an effective amount of a fusion protein comprising (1) the extracellular domain of human APO-1 and human serum albumin, (2) the extracellular domain of human APO-1 and human fibrinogen, (3) the extracellular domain of human APO-1 and hemoglobin, or (4) the extracellular domain of human APO-1 and collagen and human serum albumin.

37. The method of claim 36, wherein said Fc portion is fused to amino acid residue 173 of said extracellular domain of human APO-1.

38. The method of claim 36, wherein said Fc portion is fused to amino acid residue 171 of said extracellular domain of human APO-1.

39. The method of any of claims 36, 37, or 38, wherein said cell is affected by an HIV infection.

40. The method of any of claims 36, 37, or 38, wherein said cell is affected by an autoimmune disorder.
